(19) United States
(12) Patent Application Publication
(10) Pub. No.: US 2005/0118613 A1
(43) Pub. Date: Jun. 2, 2005

(54) Methods and kits for predicting the likelihood of successful treatment of cancer

(75) Inventor: Barry Iacopetta, Crawley (AU)

Correspondence Address: FOLEY AND LARDNER;SUITE 500, 3000 K STREET NW, WASHINGTON, DC, 20007, US

(73) Assignee: Oncomethylome Sciences S.A.

(21) Appl. No.: 10/903,859

(22) Filed: Aug. 2, 2004

Related U.S. Application Data
(60) Provisional application No. 60/492,274, filed on Aug. 1, 2003.

(51) Int. Cl. C12Q 1/68; G01N 33/574; A61K 31/52

(52) U.S. Cl. 435/6; 435/7.23; 514/263.1

(57) ABSTRACT

Methods and kits for determining the appropriate treatment for cancer, more specifically for determining the likelihood of successful treatment of cancer using antimetabolic compounds.

### CROSS-REFERENCE TO RELATED PATENT APPLICATIONS

This application claims the benefit of U.S. Provisional Application Ser. No. 60/492,274, filed Aug. 1, 2003, the entire contents of which are hereby incorporated by reference.

### FIELD OF THE INVENTION

The present invention relates to kits and methods for determining appropriate cancer therapy and tracking the likelihood of success of a particular cancer therapy for a specified cancer indicated for treatment.

### BACKGROUND OF THE INVENTION

Transcriptional silencing of tumor suppressor genes associated with the hypermethylation of CpG dinucleotide “islands” located within promoter regions is thought to be an important epigenetic mechanism for carcinogenesis (1). The simultaneous hypermethylation of multiple genes including p16, THBS1, IGF-2, and HIC-1 is referred to as CIMP+ (2, 3) and is observed in approximately 20-40% of colorectal tumors (3-5). In a proportion of these tumors, the DNA mismatch repair gene hMLH1 is hypermethylated (6, 7). This is associated with a lack of hMLH1 expression and consequently with widespread instability in microsatellite sequences, in particular large mononucleotide repeats such as BAT-26. Sporadic colorectal cancers (CRCs) with the CIMP+ or MSI+ phenotypes share several important biological features including frequent location in the proximal colon (2, 4, 5, 8-10), poor histological differentiation (4, 5, 9, 10), and wild-type p53 (3-5, 9). These common properties suggest that CIMP+ and MSI+ CRCs develop along a similar pathway, possibly involving serrated adenomas and hyperplastic polyps as precursors (11, 12). In the present study, the inventors have therefore investigated the predictive value of CIMP+ by comparing the survival of stage III CRC patients treated with or without 5-FU.

### BRIEF DESCRIPTION OF THE DRAWINGS

FIG. 1 shows prognostic values for CIMP+ in stage III CRC patients treated with surgery alone (A) or with surgery and 5-FU (B). P values shown are from the log-rank test.

FIG. 2 shows predictive values of CIMP+ (A) and CIMP− (B) for the survival benefit from 5-FU. P values shown are from the log-rank test.

FIG. 3 shows the results of the survival analysis which proves the predictive value of hMLH1 methylation for patient response to 5-FU treatment of stage I and III colorectal cancer.

A. Shows the overall survival of patients with unmethylated HMLH1 gene (unbroken line) versus methylated hMLH1 gene (broken line ---).

B. Shows the overall survival of patients with unmethylated hMLH1 gene having no chemotherapy treatment (unbroken line) versus those who have been treated using chemotherapy (broken line ----).

C. Shows the overall survival of patients with methylated hMLH1 gene having no chemotherapy treatment (unbroken line) versus those who have been treated using chemotherapy (broken line ---). Here chemotherapy clearly has a positive impact where the hMLH1 gene is methylated.

D. Shows the overall survival of patients having no chemotherapy treatment where the hMLH1 gene is unmethylated (unbroken line) versus those who have methylated hMLH1 gene (broken line ----).

E. Shows the overall survival of patients having chemotherapy treatment where the hMLH1 gene is unmethylated (unbroken line) versus those who have methylated hMLH1 gene (broken line ----). An increase in survival is clearly seen for the patients being treated by chemotherapy where the hMLH1 gene is methylated.

### DETAILED DESCRIPTION OF THE INVENTION

The present invention seeks to provide improved methods and kits for determining the appropriate treatment for cancer, more specifically for determining the likelihood of successful treatment of cancer using antimetabolic compounds.

According to a first aspect of the invention there is provided a method for predicting the likelihood of successful treatment of cancer with an antimetabolic compound comprising measuring the CIMP status of a sample obtained from a subject, whereby if the CIMP status is positive the likelihood of successful treatment is higher than if the CIMP status is negative.

The method relies on the fact that the inventors have discovered that CpG Island Methylator Phenotype (CIMP) status has a predictive value for determining the long-term survival benefit associated with chemotherapy using antimetabolic compounds. The CpG island methylator phenotype (CIMP) is observed in approximately 30% of colorectal cancer (CRC) cases and is characterized by the concurrent methylation of multiple CpG islands in tumor DNA. This phenotype (CIMP+) is more frequently observed in tumors with proximal location, microsatellite instability, and normal p53.

“Antimetabolic compound” is defined herein to include all compounds which may inhibit cancer cell metabolism, more particularly, nucleotide and DNA metabolism, even more particularly methylation metabolism, purine metabolism, and methyl group metabolism, even more particularly folate metabolism, and even more particularly folate in nucleic acid metabolism.

A “sample” in the context of the present invention is defined to include any sample in which it is desirable to test for CIMP status. In the context of the present invention the “sample” will generally be a clinical sample. The sample being used may depend on the specific cancer type that was being tested for. By way of example, in the case of diagnosing colorectal cancer a suitable colonic sample from the subject may be required. The sample may be taken from the tumour itself or may be taken from the surrounding tissue. In one embodiment the sample will be taken from the subjects lymph node.

CIMP status is defined herein to include the investigation of the methylation status of at least two CpG loci whose methylation status shows a link to cancer. Methylation is most commonly associated with CpG islands in the promoter regions of genes. Therefore, in most cases methods of detection of CIMP status will focus on this area of the relevant genes. However, the invention is not limited to the promoter regions. If the gene is methylated elsewhere and this methylation is linked to cancer, this part of the gene may be assessed in the methods of the invention for detecting the CIMP status of a subject. Furthermore, CIMP status is also known to be linked to transcriptional silencing of specific genes including hMLH1 and p16. Consequently, CIMP may show characteristic protein expression patterns. Thus CIMP status may be measured by measuring the expression of specific genes at the RNA or protein level.

Positive CIMP status (CIMP+) is thus defined to include the following:
1) The presence of 2 or more genes which are methylated at CpG dinucleotides, wherein the methylation status of the genes is linked to cancer.
2) Altered expression levels of appropriate RNA or proteins wherein the genes encoding said RNA or proteins are methylated at CpG dinucleotides, wherein the methylation status of the genes is linked to cancer.

In a most preferred embodiment the CIMP status of the patient will be measured by determining the methylation status of a panel of genes. Preferably asessment will be made in the promoter region of the genes. Preferably the panel will comprise at least two genes.

In one embodiment the panel of genes comprises the following genes: p16, MINT-2 and MDR1. The panel of genes may include other genes, provided their methylation status is linked to the incidence of cancer. These may include any or all of the following genes, which are listed by way of example and are not intended to limit the scope of the invention: THBS1, IGF-2, HIC-1 and hMLH1, p16, p15, E-cadherin, VHL, TGFβ1, TGFβ2, P130, BRAC2, NF1, NF2, TSG101, MDGI, GSTPI, Calcitonin, HIC-1, Endothelin B receptor, TIMP-2, MGMT, MLH1, MLH2 and GFAP (see WO97/46705); MGMT, DAP kinase, RASSF1A, H-cadherin, retinoic acid receptor beta, and fragile histidine triade (see WO 02/18649); TSLC1 (see WO 02/14557); SOCS-1 SOCS-2, CIS-2 (see WO02/083705); APC, DAPK, PAX5 alpha, PAX5 beta, Gata-4, Gata-5, Dab-2, inhibin α, Tiff2, and Tiff3, AP-2 α, P73, BRAC-1, RASSF-1, P14, E-cadherin, RARbeta2, TIMP3, CDH1, BRAC-1, and Tromb. Other such genes prone to hypermethylation and wherein hypermethylation is associated with cancer development are known in the art, such as these described by Suzuki et al. in Nature Genetics (2002) 31:141-149 (38).

The terms “methylation” and “hypermethylation” are used interchangeably herein. Both are defined as methylation of CpG loci within a gene sequence, most often within the promoter of a gene, whose methylation status is linked to the incidence of cancer.

In a preferred aspect of the invention the CIMP status will be considered positive if all, or at least 2, of the promoters of the panel of genes are methylated. Obviously the number of genes in the panel may be varied, but provided 2 or more of the sites show methylation this may be deemed to be sufficient for the sample to be classified as CIMP+. Alternatively at least 3, at least 4, at least 5 or at least 6 of the genes must show methylation in order to classify the particular sample as CIMP+.

Alternatively and additionally, the RNA and/or protein expression levels of certain genes may be assessed in order to determine CIMP status. In a preferred embodiment, the CIMP status is measured by determining the expression of a panel of genes at either the RNA or protein level. Preferably, the panel of genes comprises at least the following genes: p16 and hMLH1. CIMP+ is known to be associated with the transcriptional silencing of both of these genes.

The panel of genes may include any or all of the following genes, which are listed by way of example and are not intended to limit the scope of the invention: THBS1, IGF-2, HIC-1 and hMLH1, p16, p15, E-cadherin, VHL, TGFβ1, TGFβ2, P130, BRAC2, NF1, NF2, TSG101, MDGI, GSTPI, Calcitonin, HIC-1, Endothelin B receptor, TIMP-2, MGMT, MLH1, MLH2 and GFAP (see WO97/46705); MGMT, DAP kinase, RASSF1A, H-cadherin, retinoic acid receptor beta, and fragile histidine triade (see WO 02/18649); TSLC1 (see WO 02/14557); SOCS-1 SOCS-2, CIS-2 (see WO02/083705); APC, DAPK, PAX5 alpha, PAX5 beta, Gata-4, Gata-5, Dab-2, inhibin α, Tiff2, and Tiff3, AP-2 α, P73, BRAC-1, RASSF-1, P14, E-cadherin, RARbeta2, TIMP3, CDH1, BRAC-1, and Tromb. Other such genes prone to hypermethylation and wherein hypermethylation is associated with cancer development are known in the art, such as these described by Suzuki et al. in Nature Genetics (2002) 31:141-149 (38).

Suitable techniques for detecting RNA expression are well known in the art and include, for example and not by way of limitation, Northern blotting, Reverse-Transcriptase PCR (RT-PCR), Mass spectrometry and use of Microarrays. Accordingly use of these well known techniques may be incorporated in the methods of the invention.

Techniques for detecting protein expression levels include, but are not limited to, Immuno detection methods which can be broadly split into two main categories; solution-based techniques such as enzyme-linked immunosorbent assays (ELISA), immunoprecipitation and immunodiffusion, and procedures such as Western blotting and dot blotting where the samples have been immobilized on a solid support. Said methods rely on antibodies which recognize specifically the protein of interest. Said methods may be included in the methods of the present invention.

Other protein detection methods including, for example, SDS-Polyacrylamide gel electrophoresis, may be utilised in the methods of the present invention.

The method of the invention may be further enhanced in terms of sensitivity by also measuring expression levels of genes involved in folate metabolism. In a preferred embodiment these genes include any of the genes encoding thymidylate synthetase, dihydropyrimidine dehydrogenase and thymidine phosphorylase. However the invention is not intended to be limited to these specific examples, expression levels of any gene involved in folate metabolism may be measured.

Additionally, in a further aspect of the invention CIMP status may be further assessed by measuring the levels of genomic hypomethylation. Genomic hypomethylation has been shown to be associated with cancer development, mainly resulting in over-expression of certain genes in cancer tissues compared to non-cancer tissue. This hypomethylation has been observed in a variety of cancer types including pancreatic ductal adenocarcinoma, gastric and hepatocellular carcinoma, uterine leiomyoma, pilocytes astrocytomas, cervix cancers, pancreatic cancers, breast and ovarian cancers among others. Genes affected by such hypomethylation have been described and include claudin4, lipocalin2, 14-3-3sigma, trefoil factor2, S100A4, mesothelin, prostate stem cell antigen, CAGE, methyltransferases (DNMT1, 3A and 3B), MYOD1, Synuclein Gamma (SNCG, BCSG1), MUC2, H19, IGF2, CDH13, among others (see Sato et al, Cancer Res. (2002) 63:4158-4166; Cho et al. Biochem Biophys Res Commun. (2003) 307:52-63; Li et al., Gyneol Oncol. (2003) 90:123-130; Uhlmann et al., Int. J. Cancer (2003) 103:52-59; Dunn, Ann NY Acad Sci (2003) 983:28-42; Eden et al. Science (2003) 300:455, Capoa et al., Oncol. Rep. (2003) 10:545-549; Gupta et al., Cancer Res., (2003) 63:664-674; Mesquita et al., Cancer lett. (2003) 189:129-136; Cui et al. Cancer res. (2002) 62:6442-6446; Yu et al., BMC cancer (2002) 2:39).

Thus, in one embodiment CIMP status may be further determined by assessing methylation levels of any of the following genes: claudin4, lipocalin2, 14-3-3sigma, trefoil factor2, S100A4, mesothelin, prostate stem cell antigen, CAGE, methyltransferases (DNMT1, 3A and 3B), MYOD1, Synuclein Gamma (SNCG, BCSG1), MUC2, H19, IGF2, CDH13. These genes are listed solely by way of example and are not intended to be limiting with respect to the present invention. Any gene whose hypomethylation is linked to cancer may be included within the scope of the present invention.

CIMP status may be additionally determined by measuring levels of intratumoral folate intermediates, which is consistent with a disruption in folate metabolism in tumour tissues.

Thus the method of the invention may additionally incorporate measuring the levels of intratumoral folate intermediates in one particular embodiment.

The preferred techniques for use in assessing the methylation status of the panel of genes, and which may also include the assessment of genomic hypomethylation, include methylation specific PCR (MSP) or quantitative methylation specific PCR (QMSP). Both techniques will be familiar to one of skill in the art. In the MSP approach DNA may be amplified using primer pairs designed to distinguish methylated from unmethylated DNA by taking advantage of sequence differences as a result of sodium-bisulfite treatment (30). After sodium-bisulfite treatment unmethylated Cytosine's are converted to Uracil, and methylated Cytosine's remain unconverted.

An advancement of this technique is called real-time quantitative MSP (QMSP) which permits reliable quantification of methylated DNA. The method is based on the continuous optical monitoring of a fluorogenic PCR. This PCR approach can detect aberrant methylation patterns in human samples with substantial (1:10.000) contamination of normal DNA (31). Moreover, this PCR reaction is amenable to high-throughput techniques allowing the analysis of close to 400 samples in less then 2 hours without requirement for gel-electrophoresis.

Other nucleic acid amplification techniques may also be modified to detect the methylation status of the panel of genes. Such amplification techniques are well known in the art, and include methods such as NASBA (Compton, 1991 (45)), 3SR (Fahy et al., 1991 (46)) and Transcription Mediated Amplification (TMA). Amplification is achieved with the use of primers specific for the sequence of the gene whose methylation status is to be assessed. In order to provide specificity for the nucleic acid molecules primer binding sites corresponding to a suitable region of the sequence may be selected. The skilled reader will appreciate that the nucleic acid molecules may also include sequences other than primer binding sites which are required for detection of the methylation status of the gene, for example RNA Polymerase binding sites or promoter sequences may be required for isothermal amplification technologies, such as NASBA, 3SR and TMA.

TMA (Gen-probe Inc.) is an RNA transcription amplification system using two enzymes to drive the reaction, namely RNA polymerase and reverse transcriptase. The TMA reaction is isothermal and can amplify either DNA or RNA to produce RNA amplified end products. TMA may be combined with Gen-probe's Hybridization Protection Assay (HPA) detection technique to allow detection of products in a single tube. Such single tube detection is a preferred method for carrying out the invention. This list is not intended to be exhaustive, any nucleic acid amplification technique may be used provided the appropriate nucleic acid product is specifically amplified.

Thus, in a further embodiment the method of the invention is carried out using a technique selected from NASBA, 3SR and TMA.

A number of techniques for real-time detection of the products of an amplification reaction are known in the art. Many of these produce a fluorescent read-out that can be continuously monitored, specific examples being molecular beacons and fluorescent resonance energy transfer probes. Real-time techniques are advantageous because they keep the reaction in a “single tube”. This means there is no need for downstream analysis in order to obtain results, leading to more rapidly obtained results. Furthermore keeping the reaction in a “single tube” environment reduces the risk of cross contamination and allows a quantitative output from the methods of the invention. This may be particularly important in the clinical setting of the present invention. Real-time quantification of PCR reactions can be accomplished using the TaqMan® system (Applied Biosystems), see Holland et al; Detection of specific polymerase chain reaction product by utilising the 5′-3′ exonuclease activity of Thermus aquaticus DNA polymerase; Proc. Natl. Acad. Sci. USA 88, 7276-7280 (1991) (32), Gelmini et al. Quantitative polymerase chain reaction-based homogeneous assay with flurogenic probes to measure C-Erbb-2 oncogene amplification. Clin. Chem. 43, 752-758 (1997)(33) and Livak et al. Towards fully automated genome wide polymorphism screening. Nat. Genet. 9, 341-342 (19995) (34). Taqman® probes are widely commercially available, and the Taqman® system (Applied Biosystems) is well known in the art. Taqman® probes anneal between the upstream and downstream primer in a PCR reaction. They contain a 5′-fluorophore and a 3′-quencher. During amplification the 5′-3′ exonuclease activity of the Taq polymerase cleaves the fluorophore off the probe. Since the fluorophore is no longer in close proximity to the quencher, the fluorophore will be allowed to fluoresce. The resulting fluorescence may be measured, and is in direct proportion to the amount of target sequence that is being amplified.

In the Molecular Beacon system, see Tyagi & Kramer. Molecular beacons—probes that fluoresce upon hybridization. Nat. Biotechnol. 14, 303-308 (1996) (35) and Tyagi et al. Multicolor molecular beacons for allele discrimination. Nat. Biotechnol. 16, 49-53 (1998) (36), the beacons are hairpin-shaped probes with an internally quenched fluorophore whose fluorescence is restored when bound to its target. The loop portion acts as the probe while the stem is formed by complimentary “arm” sequences at the ends of the beacon. A fluorophore and quenching moiety are attached at opposite ends, the stem keeping each of the moieties in close proximity, causing the fluorophore to be quenched by energy transfer. When the beacon detects its target, it undergoes a conformational change forcing the stem apart, thus separating the fluorophore and quencher. This causes the energy transfer to be disrupted to restore fluorescence.

Any suitable fluorophore is included within the scope of the invention. Fluorophores that may possibly be used in the method of the invention include, by way of example, FAM, HEX™, NED™, ROX™, Texas Red™ etc. Quenchers, for example Dabcyl and TAMRA are well known quencher molecules that may be used in the method of the invention. However, the invention is not limited to these specific examples.

A further real-time fluorescence based system which may be incorporated in the methods of the invention is Zeneca's Scorpion system, see Detection of PCR products using self-probing amplicons and fluorescence by Whitcombe et al. Nature Biotechnology 17, 804-807 (1 Aug. 1999) (37). This reference is incorporated into the application in its entirety. The method is based on a primer with a tail attached to its 5′ end by a linker that prevents copying of the 5′ extension. The probe element is designed so that it hybridizes to its target only when the target site has been incorporated into the same molecule by extension of the tailed primer. This method produces a rapid and reliable signal, because probe-target binding is kinetically favoured over intrastrand secondary structures.

Thus, in a further aspect of the invention the products of methylation specific amplification are detected using real-time techniques. In one specific embodiment of the invention the real-time technique consists of using any one of the Taqman system, the Molecular beacons system or the Scorpion probe system.

In a most preferred embodiment the methylation status of the panel of genes is determined in a single experiment. Thus, the reaction mixture will contain all of; the sample under test, the primers and probes required to determine the methylation status of the genes, the required buffers and all reagents and enzymes required for amplification in addition to the reagents required to allow real time detection of amplification products. Thus the entire method for predicting the likelihood of successful treatment of cancer using antimetabolic compounds, occurs in a single reaction, with a quantitative output, and without the need for any intermediate washing steps. Use of a “single tube” reaction is advantageous beacuse there is no need for downstream analysis in order to obtain results, leading to more rapidly obtained results. Furthermore keeping the reaction in a “single tube” environment reduces the risk of cross contamination and allows a quantitative output from the methods of the invention. Also, single tube reactions are more amenable to automation, for example in a high throughput context.

Multiplexing (the assessment of the methylation status of a number of genes in a single tube) may be performed by using labeled primers according to the LUX™ fluorogenic primers from Invitrogen™ or as described by Nazarenko et al. NAR 30:e37 (2002) and Nazarenko et al. NAR 30:2089-2095 (2002). This technology is based on labeling and designing at least one of the primers in the primer pair in such a way that it contains a hairpin structure. A fluorescent label is attached to the same primer. Said fluorophore may be FAM or JOE, for example. The hairpin functions as a quencher. The skilled reader would appreciate that alternatives to such probes will work equally well with the invention.

Alternatively, the method of the invention may be carried out in step-wise fashion. Thus, the methylation status of each of the panel of genes may be determined in a separate experiment and the results aggregated to assess the CIMP status of the subject.

Primers specific for the genes whose methylation status was to be detected are utilised in the methods and kits of the invention. Any primer that can direct sequence specific amplification with minimum background, non-specific amplification, and can distinguish between methylated and unmethylated DNA following sodium bisulphite treatment may be utilised. Primers may comprise DNA or RNA and synthetic equivalents depending upon the amplification technique being utilised. For example, for standard PCR a short single stranded DNA primer pair tends to be used, with both primers bordering the region of interest (containing CpG motifs) to be amplified. The types of primers that may be used in nucleic acid amplification technology such as PCR (including MSP and QMSP), 3SR, NASBA and TMA, for example, are well known in the art.

Suitable probes for use in the real-time methods may also be designed, in order that they may be used in conjunction with the primers used in the methods and kits of the invention. Thus, for example, when using the Taqman technique, the probes may need to be of sequence such that they can bind between primer binding sites on the relevant gene containing sites which may be methylated in a cancer patient who is predicted to respond well to treatment using antimetabolic compounds in accordance with the present invention. Similarly molecular beacons probes may be designed that bind to a relevant portion of the nucleic acid sequence incorporated into the methods and kits of the invention. If using the Scorpion probe technique for real time detection the probe is designed such that it hybridizes to its target only when the target site has been incorporated into the same molecule by extension of the tailed primer. Therefore, the invention further provides for inclusion of probes suitable for use in real-time detection methods in the present invention.

Alternative methods of detection of the methylation status of the panel of genes may be utilised which do not depend upon the PCR reaction. Such alternative detection methods may be used independently or in combination with PCR. Examples of alternative detection techniques include mass spectrometry, including matrix assisted laser desorption (MALDI) mass spectrometry and MALDI-Time of Flight (MALDI-TOF) mass spectrometry, chromatography and use of microarray technology (Motorola, Nanogen), Reversed hybridisation and Methylation sensitive restriction enzymes (see below). With respect to a microarray, multiple suitable CpG island tags may be arrayed as templates on a solid support. The solid support may be a microchip for example. Amplicons may be prepared from test samples and also control cells (positive and negative controls). These amplicons may then be used to probe the arrays in order to detect the methylation status of the panel of genes and therefore provide the CIMP status of the subject. Mass spectrometry allows the expected molecular weight of the methylated genes to be accurately measured. MALDI-TOF relies upon a high voltage potential which rapidly extracts ions and accelerates them down a flight tube. A detector at the end of the flight tube is used to determine the time elapsed from the initial laser pulse to detection of the ions. The flight time is proportional to the mass of the ion. The accuracy of the technique allows methylated genes to be distinguished from unmethylated genes.

Restriction enzyme (RE) analysis may also be used to detect the methylation status of the panel of genes whose methylation status is linked to cancer and may therefore be used to indicate the CIMP status of a subject. Methylation of gene sequences is known to protect them from restriction enzyme digestion and so methylation may be detected by observing a change in the RE pattern for a gene sequence compared to an unmethylated control sequence.

The read out from the methods will preferably be a fluorescent read out, but may comprise, for example, an electrical read out.

The method of the invention is most preferably used to predict the likelihood of successful antifolate treatment of colorectal cancer (CRC). However, other “antimetabolic compounds” (for a definition see page 2) including anti-folate compounds, Thymidilate synthase inhibitors and other “antimetabolic compounds” have been used for treatment in cancer chemotherapy in a variety of cancer types, including but not limited to pancreatic cancers, breast cancers, prostate cancers, gastric cancers, Cervix cancers, lung cancers, esophageal cancers, Renal cancers and head and neck cancers, see Smith and Gallagher, Eur J. Cancer (2003) 39:1377-1383 (39); Droz et al., Ann. Oncol. (2003) 14:1291-1298 (40); Cocconi et al. Ann. Oncol. (2003) 14:1258-1263 (41); F. G., J. Obset. Gyneaecol. (2003) 23:422-425 (42); Focan et al., Pathol Biol (Paris) (2003) 51:204-205 (43); Lee et al., Acta Oncol. (2003) 42:207-217 (44). Because CIMP+ status may be a marker for widespread aberrations in cellular folate and methyl group metabolism, such changes may render all types of CIMP+ tumour cells more sensitive to the antifolate therapies mentioned herein, which are well known in the art. This means that the method of the invention may be applied to predict the likelihood of successful treatment for a number of different cancers using antimetabolic compounds, because CIMP+ status is likely to have similar implications for each type of cancer.

Antimetabolic compounds include inhibitors of Thymidilate synthase and other enzymes such as dihydrofolate reductase, AICAR transformylase, GAR transformylase, several methyl transferases, methylenetetrahydrofolate reductase, DNA polymerase adenosine deaminase, methionine synthase, and cystathionine-beta-synthase including both antifolate compounds and nucleotide analogues and all compounds which have an anti metabolic activity in the folate pathway and even more particularly in the folate pathway in nucleic acid metabolism, as described in more detail below.

In one aspect of the invention the antifolate which is utilised to treat the cancer is 5-fluorouracil (5-FU). 5-FU was first synthesized in 1957 and is representative of the first class of Thymidylate synthase (TS) inhibitors. Since then, a whole series of Thymidylate synthase inhibitors have been synthesized and developed, including 5-FU analogues. These compounds fall mainly into two classes: the folate analogues and the nucleotide analogues.

Anti-folate analogues, such as fluorodeoxyuridine, ftorfur, 5′-deoxyfluoruridine, raltitrexed, UFT, S-1,5-ethynyluracil, Capecitabine, pemetrexed, nolatrexed, ZD9331, trimetrexate, LU231514, edatrexate, GW1843, GW1843, OSI-7904L, Leucovorin, Levimosole, Methotrexaate, GS7904L, PDX, 10-EdAM, ICI-198,583, DDATHF and others are presently under study in the clinic. Thymydilate synthase inhibitors other than anti-folate compounds such as CB300638, 4-S-CAP, N-ac-4-S-CAP are also well known. Other such anti-folate compounds and thymidylate synthase inhibitors are known in the art (see Theti et al. Cancer res. (2003) 63:3612-3618; Ackland et al., Cancer Chemother Biol Response modif. (2002) 20:1-36; Pawelczak et al, Act Biochim Pol. (2002) 49:407-420; Chu et al. Cancer Chemother. Pharmacol. (2003) 52 supl 1:80-89; Wang et al. Leuk lymphoma. (2003) 44(6):1027-1035; Van Der Laan et al., Int. J. Cancer (1992) 51:909-914; Papamichael, Stem Cell. (2000) 18:166-175; Prezioso, et al., Cancer chemother. Pharmacol. (1992) 30:394-400; Ismail et al., Cancer Chemother Biol response Modif. (2001) 19:1-19).

The inventors have clearly shown that 5-FU chemotherapy in cancer patients having hypermethylated genes has more clinical benefit or gives better response to therapy than subjects having no such hypermethylated genes. Other anti-folate compounds, and other thymydilate synthase inhibitors, including but not limited to those mentioned above, will also result in more clinical benefit or result in a better response to therapy in cancer patients having hypermethylated genes, than subjects lacking hypermethylation in the appropriate genes.

5-FU and other anti-folate compounds are known to prevent or inhibit methylation of DNA. However, even after many years of use in clinical practice, the exact mode of action for both 5-FU and other anti-folates is still debatable, but is largely considered as a thymidylate synthase (TS) inhibitor. More particularly such compounds have an anti-metabolic activity in the folate pathway and even more particularly in the folate pathway in nucleic acid metabolism. This pathway is critical in de novo cellular purine nucleotide biosysnthesis and DNA methylation. Enzymes involved include, in addition to Thymidilate synthase other enzymes such as dihydrofolate reductase, AICAR transformylase, GAR transformylase, several methyl transferases, methylenetetrahydrofolate reductase, among others. Some key intermediates and vitamins that play a key role in these pathways are methionine, choline, vitamin B-6, vitamin B-12, riboflavin (vitaminB-2), S-adenosylmethionine, homocysteine, S-adenosylhomocysteine, methyl malonic acid, tetrahydrofolate, dihydrofolate, among others (see Potter, J. nutr. (2002)132 (8 Suppl.):2410S-2412S; Mason et al. J. nutr. (2002) 133(Suppl. 3):941S-947S; Plasche et al. Cancer Lett. (2003) 191:179-185; Choi et al, (2000) J. Nutr. 130:129-132). Compounds known to inhibit these and other metabolic pathways including the thymidilate synthase pathway, the the purine biosynthesis pathway, the methyl metabolism pathway, the DNA synthesis pathway have been used successfully in cancer chemotherapy. They include, by way of example and not limitation with respect to the present invention; Cytarabine (Ara-C) and Gemcitabine which interfere with DNA polymerase, 6-MP and 6-TG (thiopurines), which cause strand breaks when incorporated into DNA, Fluarabine which also causes strandbreaks, and in addition is an inhibitor of DNA polymerase and RNA polymerase function, Cladribine which can cause strand breaks in the nucleic acid of subjects suffering from leukemia's, and Pentostatin which inhibits the Adenosine deaminase (RR) enzyme and halts DNA synthesis. Such compounds are included within the scope of the present invention.

The inventors have clearly shown that 5-FU chemotherapy in cancer patients having hypermethylated genes has more clinical benefit or gives better response to therapy than subjects lacking hypermethylation in the same genes. Other antimetabolics, including but not limited to these mentioned above, will also result in more clinical benefit or result in a better response to therapy in cancer patients having hypermethylated genes, than subjects lacking hypermethylation in these genes.

As mentioned above, a “sample” in the context of the present invention is defined to include any sample in which it is desirable to test for CIMP status. In the context of the present invention the “sample” will generally be a clinical sample. The sample being used may depend on the specific cancer type that was being tested for. By way of example, in the case of diagnosing colorectal cancer a suitable colonic sample from the subject may be required. The sample may be taken from the tumour itself or may be taken from the surrounding tissue. In one embodiment the sample will be taken from the subjects lymph node.

The sample may be obtained from any body fluid of the subject provided it contained the markers (genes and/or RNA and/or proteins) necessary to assess CIMP status of the subject. For example, a blood sample may be utilised, provided the appropriate markers to allow analysis of CIMP status are present in the sample. Typical samples which may be used, but which are not intended to limit the invention, include whole blood, serum, plasma, urine, chyle, stool, ejaculate, sputum, nipple aspirate, saliva etc. taken from a subject, most preferably a human subject.

In a most preferred embodiment the test will be an in vitro test carried out on a sample removed from the subject.

In a further embodiment the above-described methods may additionally include the step of obtaining the sample from the subject. Methods of obtaining a suitable sample are well known in the art. Alternatively, the method may be carried out beginning with a sample that has already been isolated from the subject in a separate procedure. The methods are most preferably carried out on a sample from a human, but the method of the invention may have diagnostic utility for many animals.

The present invention also provides a method of selecting a suitable treatment regimen for cancer comprising determining the CIMP status of a sample obtained from a subject, whereby if the CIMP status is positive chemotherapy using antimetabolics may be administered to the subject. Said chemotherapy may be utilised optionally in conjunction with surgical techniques.

On the other hand, if the CIMP status is negative, surgical techniques may be utilised, possibly in conjunction with other chemotherapies, other than treatment using antimetabolic compounds, which is unlikely to be a suitable method of treatment if the CIMP status is negative (CIMP−).

The method for selecting a suitable treatment regimen may incorporate all of the optional features described for the methods of predicting the likelihood of successful treatment of cancer with an antimetabolic compound comprising measuring the CIMP status of a sample obtained from a subject.

The inventors have clearly shown that antimetabolic chemotherapy in subjects suffering from cancer has more clinical benefit or gives better response to therapy for patients having hypermethylated genes than patients lacking hypermethylation in these genes. Therefore, by measuring CIMP status, a specific subgroup of cancer patients who are more likely to respond to antimetabolic chemotherapy can be identified. The CIMP status of a subject acts as an accurate indicator leading to treatment of patients with all antimetabolic compounds (specific examples of which are given herein but are not intended to limit the scope of the invention.)

Present indications for the treatment of cancer patients with chemotherapies is mainly based on the origin (colon, breast prostate, cervix, etc.) or the histological characterization of the cancer (carcinoma, sarcoma, myeloma, leukemia, lymphoma, etc.). The inventors have introduced a new indication for cancer patients based on the CIMP status of the patients, which allows successful treatment of the subject in need of treatment using antimetabolic compounds.

Accordingly, in a further embodiment of the invention, there is provided the use of antimetabolic compounds in the treatment of a subject suffering from cancer, wherein said subject has a positive CIMP status.

Furthermore, also provided are antimetabolic compounds for use in the manufacture of a medicament for the treatment of a subject suffering from cancer, wherein said subject has a positive CIMP status.

Additionally, also provided is a method of treating a subject suffering from cancer comprising administering antimetabolic compounds, wherein said subject has positive CIMP status.

The cancer, as mentioned above may be selected from any cancer, more particularly is selected from colorectal cancer (CRC), pancreatic cancers, breast cancers, prostate cancers, gastric cancers, Cervix cancers, lung cancers, esophageal cancers, Renal cancers, head and neck cancers.

Most preferably., the cancer is colorectal cancer (CRC).

All of the optional features described above, particularly the examples of suitable antimetabolic compounds are incorporated into these embodiments of the invention.

The invention also provides kits which may be used in order to carry out the methods of the invention. The kits may incorporate any of the preferred features mentioned in connection with the methods of the invention above.

Thus in a further aspect the invention provides a kit for predicting the likelihood of successful treatment of cancer with antimetabolic compounds comprising
means for measuring the CIMP status of a sample obtained from a subject; and
means for contacting the sample with the means for measuring CIMP status.

By providing the necessary means for determining the CIMP status of a sample (as defined above), the kit allows an appropriate treatment regimen for the specific cancer to be selected.

In a most preferred embodiment the means for measuring the CIMP status of a sample includes means for determining the methylation status of the promoters of a panel of genes. The panel of genes may be the same as that described for the methods of the invention above.

Since a preferred method of detecting the methylation status of a panel of genes utilises MSP and/or QMSP, in a preferred embodiment the kits of the invention will include suitable MSP and/or QMSP reagents. Such reagents are well known in the art and include, for example, DNA isolation reagents, polymerase enzymes for amplification, sodium bisulphite, MSP/QMSP specific buffers etc.

DNA isolation reagents are needed in order to purify DNA from samples, which may be any sample type containing suitable genes in order to detect CIMP status. Such DNA isolation reagents are well known in the art, for example phenyl-chloroform extraction is a commonly used technique. Kits may include phosphate buffered saline (PBS) for suspending cells and wash buffer (10 mM HEPES-KOH (pH=7.5); 1.5 mM MgCl₂; 10 mM KCl; 1 mM dithiothreitol). DNA may be extracted using standard salt-chloroform techniques and therefore such reagents may be included in the kits of the invetion. Ethanol precipitation may be used to obtain high molecular weight DNA, and such reagents used in this technique may be included within the scope of the invention. TE buffer (10 mM Tris; 1 mM EDTA (pH 8.0)) may also be included for dissolving DNA samples. Alternatively, for example, distilled water may be used.

As both the MSP and QMSP techniques are well known in the art such buffers and enzymes will be familiar to a person of skill in the art.

Primers are included in the kits of the invention that amplify the region of the genes that will be affected by sodium bisulphite treatment depending upon the methylation status of the genes (namely CpG loci). The primers will be gene specific and thus their sequence will depend upon the panel of genes that have been selected for use in determining the CIMP status of the sample from the subject. In one embodiment the panel of genes will include the genes p16, MINT-2 and MDR1 and thus the primers will be of specific sequence to determine the methylation status of these genes.

Kits of the invention may also include further components necessary for the MSP and/or QMSP reaction. Thus, reagents are required for the sodium bisulphite treatment of the extracted DNA. Also required are PCR enzymes, such as Taq polymerase in order to amplify the DNA sequences. As the MSP and QMSP techniques are well known in the art the reagents neccessary for their implementation will also be well known to one of skill in the art. Any such reagents are included in the scope of the present invention.

Similarly other amplification techniques, such as 3SR, NASBA and TMA are well known in the art. Kits containing suitable primers, probes and reagents to allow use of these techniques are within the scope of the present invention.

A kit may also be provided which allows RE analysis of CIMP status. As aforementioned methylation of gene sequences is known to protect them from digestion by many restriction enzymes, well known in the art, and so methylation may be detected by observing a change in the RE pattern for a gene sequence compared to an unmethylated control sequence. Thus the kit may include suitable restriction enzymes and buffers, and possibly means, such as markers for use in gel electrophoresis for detecting the CIMP status of a subject using RE analysis. Such restriction enzymes are widely commercially available and in most cases are provided with an appropriate buffer. Similarly suitable means for assessing RE digestion patterns, such as gel electrophoresis, are well known in the art.

Probes may also be included in the kits of the invention to allow real time detection of amplification products. In a preferred embodiment the kit will contain gene specific probes and reagents to allow real-time detection of QMSP reaction products. In a preferred aspect the real-time detection method is selected from Taqman system, Molecular beacons system and Scorpion probe system. Thus the kit may contain suitable reagents to allow each of these methods to be utilised. The probes are accordingly different depending on which real time detection method was being utilised. For example, when using Taqman probes or molecular beacons the probes may contain a fluorescer and a quencher at opposite ends such that they bind in between the primers that amplify the methylated region of the gene. In the Scorpion system the probe element is designed so that it hybridizes to its target only when the target site has been incorporated into the same molecule by extension of the tailed primer.

Any suitable fluorophore is included within the scope of the invention. Fluorophores that may possibly be included in the kits of the invention include, by way of example, FAM, HEX™, NED™, ROX™, Texas Red™ etc. Similarly the kits of the invention are not limited to a single quencher. Quenchers, for example Dabcyl and TAMRA are well known quencher molecules that may be used in the method of the invention and included in the kits of the invention.

Kits of the invention may also include further components necessary for the generation and detection of PCR products other than those described above, such a microarrays, which may be used for detection of PCR products, or may be used to amplify (PCR on chip) and detect the PCR product. Other components may further include “micro fluid cards” as described by Applied Biosystems, Reversed hybridization strips such as those described by LIPA technology (Innogenetics, Zwijnaarde, Belgium, or as those described by Ulysis and ULS technology (Kreatech Biotechnologies, Amsterdam, The Netherlands). Such components are known in the art and are listed by way of example and not limitation, for inclusion in the kits of the invention.

Because CIMP status may be measured also at the level of RNA and protein expression, kits are provided which allow determination of CIMP status by measuring the expression of a panel of genes at either the RNA or protein level. The panel of genes may include any genes whose methylation status is linked to the incidence of the cancer under study. Suitable examples are listed above in relation to the methods of the invention. In one embodiment the panel of genes includes p16 and hMLH1 (either alone or in combination with other genes).

Suitable techniques for detecting RNA expression are well known in the art and include, for example and not by way of limitation, Northern blotting, Reverse-Transcriptase PCR (RT-PCR), Mass spectrometry and use of Microarrays. Accordingly suitable reagents for use of these well known techniques may be incorporated in the kits of the invention.

Techniques for detecting protein expression levels include, but are not limited to, Immuno detection methods which can be broadly split into two main categories; solution-based techniques such as enzyme-linked immunosorbent assays (ELISA), immunoprecipitation and immunodiffusion, and procedures such as Western blotting and dot blotting where the samples have been immobilized on a solid support. Said methods rely on antibodies which recognize specifically the protein of interest. For the kits of the invention suitable antibodies may be included which recognize the protein expressed from those genes whose methylation status is linked to the incidence of the cancer type of interest. Furthermore, suitable buffers and reagents may also be incorporated into the kits of the invention. These may include, for example, non-specific binding blocker buffers (such as BSA, 1%, in TBST), nitrocellulose or PVDF membranes, TBS, methanol and/or ethanol, a secondary antibody conjugated to an enzyme, such as alkaline phosphatase or horseradish peroxidase, to allow detection of primary antibody binding to the substrate.

Other protein detection methods include, for example, SDS-Polyacrylamide gel electrophoresis. In this case the kits may include reagents and buffers neccessary to run the gel, and stains for the gel, such as, for example, Coomassie Blue (Promega).

The invention will be further understood with reference to the following examples, together with the accompanying tables and figures in which:

### EXAMPLES

Table 1 shows the clinical, pathological, and molecular features of the patient cohorts treated by surgery alone or by surgery and 5-FU.

Table 2 shows the associations between CIMP+ and clinicopathological or molecular features.

Table 3 gives a sensitivity assessment for the predictive value of CIMP+.

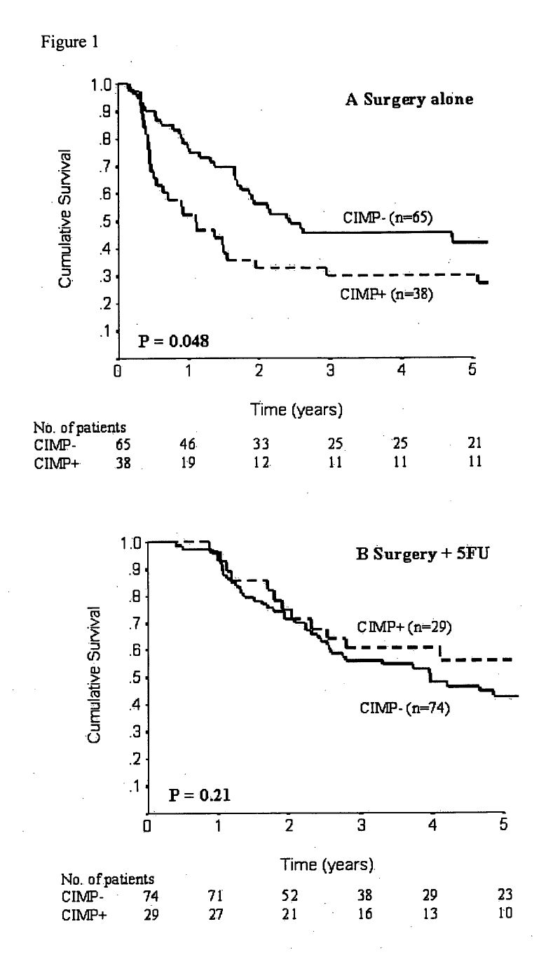

FIG. 1 shows prognostic values for CIMP+ in stage III CRC patients treated with surgery alone (A) or with surgery and 5-FU (B). P values shown are from the log-rank test.

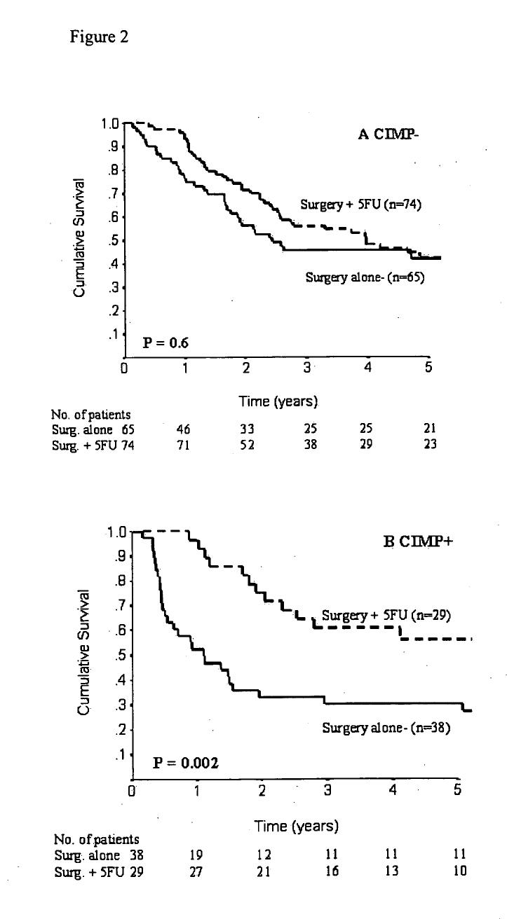

FIG. 2 shows predictive values of CIMP+ (A) and CIMP− (B) for the survival benefit from 5-FU. P values shown are from the log-rank test.

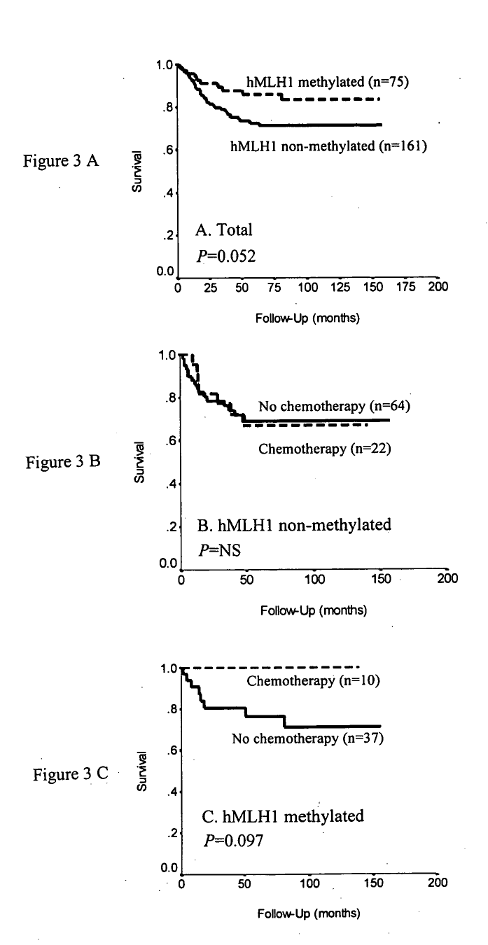

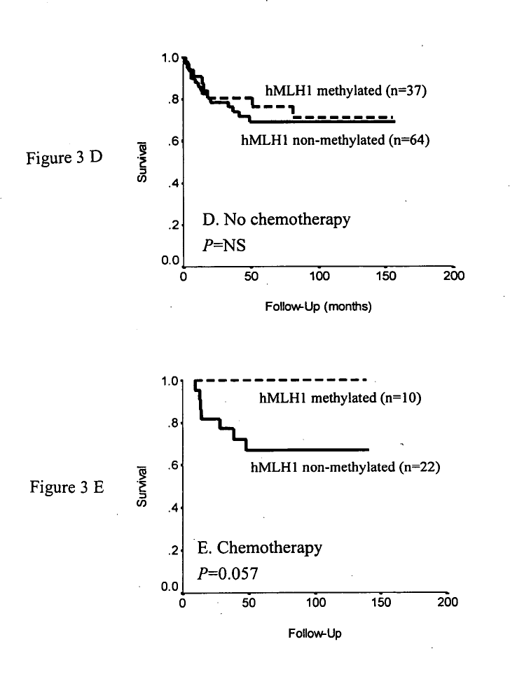

FIG. 3 shows the results of the survival analysis which proves the predictive value of hMLH1 methylation for patient response to 5-FU treatment of stage II and III colorectal cancer.

A. Shows the overall survival of patients with unmethylated hMLH1 gene (unbroken line) versus methylated HMLH1 gene (broken line ---).

B. Shows the overall survival of patients with unmethylated hMLH1 gene having no chemotherapy treatment (unbroken line) versus those who have been treated using chemotherapy (broken line ----).

C. Shows the overall survival of patients with methylated HMLH1 gene having no chemotherapy treatment (unbroken line) versus those who have been treated using chemotherapy (broken line ----). Here chemotherapy clearly has a positive impact where the HMLH1 gene is methylated.

D. Shows the overall survival of patients having no chemotherapy treatment where the hMLH1 gene is unmethylated (unbroken line) versus those who have methylated HMLH1 gene (broken line ----).

E. Shows the overall survival of patients having chemotherapy treatment where the hMLH1 gene is unmethylated (unbroken line) versus those who have methylated hMLH1 gene (broken line ----). An increase in survival is clearly seen for the patients being treated by chemotherapy where the hMLH1 gene is methylated.

Patients and Methods

Tumor Series.

A total of 891 stage III CRC cases were diagnosed at the Sir Charles Gairdner Hospital between 1985 and 1999 (14). This spans the time period during which 5-FU-based adjuvant chemotherapy was being introduced in Western Australia for the management of stage III CRC. Adjuvant chemotherapy was given to 270 (30%) patients according to the standard Mayo regimen (5-FU/leucovorin). This comprised at least two cycles of chemotherapy, and for the majority of patients the full six cycles were completed. Patients were separated into categories based on 5-year age intervals, gender, and site of tumor origin. The latter two factors have been shown to influence the survival benefit from 5-FU in CRC (13, 16). Within these groups, adjuvant-treated and nontreated patients were pair-matched at random. A total cohort of 125 matched pairs was selected for DNA methylation analysis. All tumors had negative surgical margins, and patients showed no signs of metastatic disease at the time of surgery. All cases were diagnosed at a single pathology laboratory (Hospital and University Pathology Service/Pathcenter) associated with the Sir Charles Gairdner Hospital. This laboratory maintained relatively constant reporting practices during the 1985-1999 study period. Five cases were classified as T4 lesions, and all others were classified as T3. The study included 48 rectal, 24 sigmoid, 24 descending colon, 17 transverse colon, 47 ascending colon, and 46 cecal tumors. Four patients with rectal cancer received post-operative radiotherapy. Disease-specific survival information was obtained on all 206 patients by examination of hospital and West Australian Health Department records. The median follow-up time was 39 months (range, 1-172 months), with 119 patients (58%) dying as a result of recurrent disease by the end of the study. Survival data for 19 (9%) patients who died from other causes were censored at the time of death. It has been estimated that net migration out of the state of Western Australia is 0.4% per year, equating to approximately 1 case/year of the 206 cases investi-gated in this series. However, this rate is expected to be considerably lower for older individuals and particularly for those diagnosed with cancer. The Sir Charles Gairdner Hospital Human Research Ethics Committee gave approval for this study.

CIMP+ Molecular Analysis.

Toyota et al. (2) have suggested that investigation of between two and four type “C” (cancer-specific) CpG loci is sufficient for the accurate evaluation of the CIMP+ phenotype. Methylation-specific PCR was used to determine the methylation status of CpG islands located within the p16 promoter (4, 5, 10, 17), the MINT-2 clone (3, 4), and the MDR1 promoter (4, 9). DNA amplification of all three CpG loci was successful for 103 matched pairs, equating to an overall success rate of approximately 90%. CIMP+ was arbitrarily defined as the presence of two or more of these sites showing methylation. Of the 206 tumors successfully analyzed in this study for CIMP+ status, the majority (83%) were sourced from formalin-fixed and paraffin-embedded archival tissue blocks. The remaining cases were in the form of unfixed tissue samples taken shortly after surgical resection and stored frozen at −80° C.

The inventors have previously evaluated the MSI+ and p53 mutation status of the tumors included in this study (14). MSI+ status was determined by screening for deletions in the BAT-26 mono-nucleotide repeat (18), whereas screening for p53 mutations inexons 5-8 inclusive was performed by single-strand conformational polymorphism analysis (19).

Statistical Analyses.

Multivariate Cox proportional hazard test with matched-pair stratification and Kaplan-Meier analyses were used to evaluate differences in survival between patient groups. Regression sensitivity was determined by analyzing the effect of unmeasured binary confounders as described by Lin et al. (20) Statistical analyses were performed using the Stata 7.0 (College Station, Tex.) software package. All Ps are two-sided.

Results

The clinical, pathological, and molecular features of the patient cohorts treated by surgery alone or by surgery and 5-FU are shown in Table 1. There are no significant differences between these groups, with the exception of the year of surgery.

All patients who received chemotherapy were diagnosed after 1989, whereas 17 (16%) of the patients treated by surgery alone were diagnosed between 1985 and 1989. Methylation of p16, MDR1, and MINT-2 was detected in 36%, 25%, and 38% of tumors, respectively. Using a definition of two or more sites showing methylation, 33% of tumors (67 of 206 tumors) in this series were classified as CIMP+. This phenotype was significantly associated with proximal location in the colon, poor histological grade, MSI+, and normal p53 status, but not with age, gender, or extent of nodal involvement (Table 2). Of the 67 CIMP+ tumors, 21 (31%) were of poor histological grade compared to 20/136 (15%) for the CIMP− tumors (P=0.005). Similarly, 20/65 (31%) CIMP+ tumors were MSI+compared to only 8/128 (6%) CIMP− tumors (P<0.0001).

The prognostic value of CIMP+ is shown in FIG. 1 for each of the two treatment cohorts. For patients treated by surgery alone (FIG. 1A), CIMP+ was associated with worse prognosis compared to CIMP− (RR=1.65; 95% CI: [1.00-2.72]; P=0.05). However a trend for better survival of CIMP+ patients was observed in the cohort treated with surgery plus 5-FU (FIG. 1B), possibly due to an interaction between CIMP+ and chemotherapy as described below.

In agreement with results from randomized clinical trials (16, 21) the absolute 5-year survival benefit associated with the use of 5-FU in this study was approximately 11% (RR=0.62; 95% CI: [0.43-0.90]; P=0.012). When analyzed according to CIMP+ status, almost all of the long-term benefit from 5-FU treatment was attributable to the CIMP+ patient group (FIG. 2B), with no long-term survival benefit apparent for CIMP− patients (FIG. 2A; RR=0.96; 95% CI: [0.62-1.49]; P=0.86). Multivariate analysis for the matched pairs revealed that CIMP+ was predictive for survival benefit independently of MSI+ and p53 mutation status (RR=0.22; 95% CI: [0.06-0.84]; P=0.027). Neither MSI+nor TP53 status were found to have independent predictive value in a multivariate analysis model that included CIMP+ (results not shown).

Sensitivity analyses revealed that an unmeasured, high-risk confounding factor may only account for the predictive value associated with CIMP+ if it was present with at least twice the frequency in the cohort treated by surgery alone compared to that treated with 5-FU (Table 3). The relative risk associated with this confounding factor would also need to be greater than 3.0.

Discussion

Clinical trials have established that adjuvant chemotherapy with 5-FU-based regimes is associated with small but significant improvements in the survival of stage III CRC patients (21). Until recently, there was no evidence to suggest that different subgroups of CRC patients defined by gender or anatomical location of the tumor obtain different benefit from this treatment. However, data from two recent publications, one a retrospective cohort study (13) and the other a prospective, randomized study (16), indicate that the level of survival benefit from 5-FU may vary according to gender and tumor site. Female patients and patients with colon tumors appear to derive more benefit than male and rectal cancer patients, respectively. The underlying molecular basis for this differential response to 5-FU is not known.

In the current study, we therefore investigated the predictive value of CIMP+ by comparing the survival of two age-, sex-, and site-matched patient cohorts: one treated by surgery alone; and the other treated by surgery and 5-FU/leucovorin chemotherapy. 5-FU-based chemotherapy for stage III CRC was introduced over a relatively short time period during the early to mid-1990s in Western Australia, and therefore patients in adjuvant treated and nontreated cohorts were likely to have received comparable surgical procedures, pathological diagnosis, and postsurgical management. In support of this, the survival rate for patients treated by surgery alone in the early period (1985-1992) was not significantly different from that of more recent patients (1993-1999). As shown in Table 1, the two treatment cohorts also demonstrated similar clinicopathological and molecular characteristics. The absolute survival benefit associated with 5-FU treatment in this study, 11% after 5-years of follow-up, is similar to that reported for randomized clinical trials (16, 21). Although a consensus has yet to be reached for the classification of CIMP+, the definition used in the present work identified a tumor subgroup with characteristics similar to those reported by other workers (2-5, 8, 9). These include associations with proximal tumor location, poor histological grade, wild-type p53, and MSI+ (Table 2).

The prognostic value of CpG island methylation has been investigated previously. Liang et al. (22) studied 84 stage III CRC patients and found an association between p16 methylation and shortened survival. Also, a recent study of 426 cases of stage I-IV CRC reported that patients with CIMP+ tumor have worse prognosis (5). However, two other reports did not find prognostic value for p16 methylation (23) or CIMP+ (4). In the present work, the inventors observed that CIMP+ was associated with worse survival for patients treated with surgery alone, but not for patients treated with surgery and chemotherapy. Patient treatment information should therefore always be considered when interpreting data on molecular prognostic markers. The present investigation is the first to report on the predictive value of CIMP+. The novel finding of the present study is that CRC patients with CIMP+ tumors may account for the majority and perhaps all of the long-term survival benefit associated with the use of 5-FU chemotherapy (FIG. 2). The predictive significance of CIMP+ was independent of two other molecular markers, MSI+ and p53, that also have predictive value for survival benefit from 5-FU in CRC (14, 15, 24).

Sensitivity analyses revealed that unidentified confounder variables are unlikely to explain the association between CIMP+ and apparent survival benefit from 5-FU (Table 3). Statistical evaluation of unmeasured binary confounding variables has previously been used to estimate the benefit from 5-FU chemotherapy in elderly, stage III CRC patients (25). It should be noted that approximately 40% of patients with CIMP+ tumors died from recurrent CRC despite the use of 5-FU (FIG. 2B), indicating that this phenotype is not entirely specific for the prediction of response to treatment. The use of other combinations of CpG islands to define CIMP+ may yield stronger predictive information than that observed with the current panel of p16, MINT-2, and MDR1. Additional predictive factors might also be the level of expression of genes involved in 5-FU metabolism, including thymidylate synthetase, dihydro-pyrimidine dehydrogenase, and thymidine phosphorylase (26-28). The levels of genomic hypomethylation or of intratumoral folate intermediates may also be associated with the degree of response to antifolate therapies.

An alternate approach is to carry out molecular screening for CIMP+ in archival tumor specimens from previous clinical trials of 5-FU. CIMP+ is associated with the transcriptional silencing of specific genes including hMLH1 and p16, and consequently this phenotype may show characteristic protein expression patterns. If these can be accurately identified, it may allow immunohistochemical analysis of gene expression as an alternative to DNA analyses to identify the CIMP+ subgroup of CRC. Strong links have been demonstrated between folate metabolism and changes in DNA methylation (29).

The inventors hypothesize that the DNA hypermethylation observed in CIMP+ tumors may be a surrogate marker for more widespread aberrations in cellular folate and methyl group metabolism. Such changes might render CIMP+ tumor cells more sensitive to antifolate therapies including 5-FU and leucovorin. Comparison of the level of folate intermediates between CIMP+ and CIMP− tumors may shed more light on this possibility. Another explanation for the apparent chemosensitivity of CIMP+ tumors is that the transcriptional silencing associated with this phenotype inactivates genes required for cell survival in the presence of 5-FU. Proximal (13) and colonic (16) tumors appear to gain the majority of survival benefit observed from 5-FU in CRC patients. In the present study of 206 cases, 48% of proximal tumors were CIMP+ compared with only 14-15% of distal colon or rectal tumors (Table 2). In a recent study of 417 consecutive stage I-IV CRC cases, 37% of proximal tumors compared with only 9% of distal tumors were classified as CIMP+ using a definition of 3 or more CpG sites methylated out of 5 examined (5). The tumor site difference in CIMP+ frequency becomes even greater (8-fold) if only heavy methylation (3 of 3 sites methylated) is considered (4). In addition to proximal tumor location, the inventors have also shown that females appear to gain more benefit from 5-FU than males (13).

Previous studies have shown that p16 methylation (10), heavy methylation (4), and methylation of β3 of 5 CpG sites (5) are all more common in tumors from female patients. In the current study using a definition of β2 of 3 sites methylated for CIMP+, the inventors did not find a gender difference in CIMP+frequency (Table 2). However, this may be due to the selected nature of the current patient cohort in comparison with nonselected series used in previous studies. In particular, the median age of patients in this study was 7 years younger than that seen in a large consecutive series from our institute (14). In conclusion, the present study provides evidence that CIMP+ is a predictive factor for survival benefit from 5-FU chemotherapy in CRC patients independently of MSI+ and p53 status. Confirmation of these findings may lead to the improved selection of CRC patients to receive adjuvant 5-FU chemotherapy.

The observed correlation between higher CIMP+ frequency in proximal tumors and greater survival benefit from chemotherapy is suggestive of a causal link. Comparisons of the cellular folate pool and of gene expression patterns between CIMP+ and CIMP− tumors may help to explain the apparent chemosensitivity of tumors with aberrant DNA methylation.

TABLE 1
Characteristics of CRC patients in the two treatment cohorts.

| Feature (n) | Surgery (%) | Surgery + 5FU (%) | P value |
|---|---|---|---|
| Total (206) | 103 | 103 | |
| 1. Mean age (years) | 61.4 | 60.4 | 0.48 |
| Mean follow-up (months) | 44.3 | 47.9 | 0.54 |
| Year of surgery | | | |
| 1985-1989 (17) | 17 | 0 | <0.01 |
| 1990-1994 (106) | 58 | 48 | |
| 1995-1999 (83) | 28 | 55 | |
| Sex | | | |
| female (74) | 37 (50) | 37 (50) | 1 |
| male (132) | 66 (50) | 66 (50) | |
| Site | | | |
| rectum (49) | 24 (49) | 25 (51) | 1 |
| distal colon (47) | 24 (51) | 23 (49) | |
| proximal (110) | 55 (50) | 55 (50) | |
| Histological grade | | | |
| well/moderate (162) | 78 (48) | 84 (52) | 0.36 |
| poor (41) | 23 (56) | 18 (44) | |
| Nodal involvement | | | |
| 1 or 2 nodes (96) | 44 (46) | 52 (54) | 0.26 |
| ³ 3 nodes (110) | 59 (54) | 51 (46) | |
| p53 | | | |
| wild-type (110) | 51 (46) | 59 (54) | 0.17 |
| mutant (76) | 43 (57) | 33 (43) | |
| MSI | | | |
| −165 | 79 (48) | 86 (52) | 0.62 |
| + (28) | 12 (43) | 16 (57) | |
| CIMP | | | |
| −139 | 65 (47) | 74 (53) | 0.18 |
| + (67) | 38 (57) | 29 (43) | |

TABLE 2
Associations between CIMP+ and clinicopathological or molecular
features.

| Feature (n) | CIMP− (%) | CIMP+ (%) | P value |
|---|---|---|---|
| total (206) | 139 (67) | 67 (33) | |
| sex | | | |
| female (74) | 50 (68) | 24 (32) | 0.98 |
| male (132) | 89 (67) | 43 (33) | |
| age^{†} | | | |
| <62 years (105) | 70 (67) | 35 (33) | 0.8 |
| ³ 62 years (101) | 69 (68) | 32 (32) | |
| site | | | |
| rectum (49) | 42 (86) | 7 (14) | <0.005 |
| distal colon (47) | 40 (85) | 7 (15) | |
| proximal (110) | 57 (52) | 53 (48) | |
| grade | | | |
| well/moderate | 116 (72) | 46 (28) | <0.005 |
| poor (41) | 20 (49) | 21 (51) | |
| nodal involvement | | | |
| 1 or 2 nodes (89) | 59 (66) | 30 (34) | 0.9 |
| ³ 3 nodes (94) | 63 (67) | 31 (33) | |
| p53 | | | |
| wild-type (110) | 65 (59) | 45 (41) | <0.005 |
| mutant (76) | 61 (80) | 15 (20) | |
| MSI | | | |
| negative (165) | 120 (73) | 45 (27) | <0.005 |
| positive (28) | 8 (29) | 20 (71) | |

†median age of patients was 62 years

TABLE 3
Sensitivity assessment for the predictive value of CIMP+

| Prevalence of | Prevalence of UBC | | Predictive value of |
|---|---|---|---|
| UBC in surgery | in surgery + 5-FU | | CIMP+ adjusted |
| alone cohort (%) | cohort (%) | UBC RR | for UBC |
| 0 | 0 | 1 | 0.22 [0.06-0.84] |
| 40 | 20 | 3 | 0.28 [0.08-1.00] |
| 90 | 50 | 3 | 0.30 [0.08-1.09] |
| 40 | 20 | 2 | 0.25 [0.07-0.91] |
| 90 | 50 | 2 | 0.27 [0.08-0.98] |

UBC = unmeasured binary confounder;
RR = relative risk

Data on Predictive Value of hMLH1 Methylation.

The predictive value of hMLH1 methylation for survival benefit with 5FU chemotherapy was examined in a series of stage II and III colorectal cancers that had been treated by surgery alone (no chemotherapy) or by surgery plus standard 5FU chemotherapy.

The following primers were used to detect methylated hMLH1 following bisulfite conversion (utilising the MSP technique):

| | |
|---|---|
| Forward 5′-TTAATAGGAAGAGCGGATAGC-3′ | (SEQ ID NO:1) |
| Reverse 5′-CTATAAATTACTAAATCTCTTCG-3′ | (SEQ ID NO:2) |

The sample sizes were as follows:

| | |
|---|---|
| hMLH1 non-methylated cases | N = 86 |
| hMLH1 methylated cases | N = 47 |

The average follow-up time for patients was as follows:

| | |
|---|---|
| Surgery alone (no chemotherapy) | 64 months (range 0.5-185 months) |
| Surgery plus chemotherapy | 71 months (range 9-203 months) |

The survival of patient groups was as follows:

| | |
|---|---|
| hMLH1 non-methylated/no chemotherapy | 48/64 (75%) |
| hMLH1 non-methylated/chemotherapy | 15/22 (68%) P = NS |
| hMLH1 methylated/no chemotherapy | 29/37 (78%) |
| hMLH1 methylated/chemotherapy | 10/10 (100%) P = 0.097 |

### Conclusion

The data on hMLH1 methylation supports the previous results using p16, MINT-2 and MDR1 showing that tumors in which specific genes are methylated are responsive to 5FU chemotherapy.

### REFERENCES

1. Jones, P. A., and Laird, P. W. Cancer epigenetics comes of age. Nat. Genet., 21:163-167,1999.
2. Toyota, M., Ahuja, N., Ohe-Toyota, M., Herman, J. G., Baylin, S. B., and Issa, J. P. CpG island methylator phenotype in colorectal cancer. Proc. Natl. Acad. Sci. USA, 96: 8681-8686, 1999.
3. Toyota, M., Ohe-Toyota, M., Ahuja, N., and Issa, J. P. Distinct genetic profiles in colorectal tumors with or without the CpG island methylator phenotype. Proc. Natl. Acad. Sci. USA, 97: 710-715, 2000.
4. Van Rijnsoever, M., Grieu, F., Elsaleh, H., Joseph, D., and Iacopetta, B. Characterisation of colorectal cancers showing hypermethylation at multiple CpG islands. Gut, 51: 797-802, 2002.
5. Hawkins, N., Norrie, M., Cheong, K., Mokany, E., Ku, S. L., Meagher, A., O'Connor, T., and Ward, R. CpG island methylation in sporadic colorectal cancers and its relationship to microsatellite instability. Gastroenterology, 122: 13761387, 2002.
6. Kane, M. F., Loda, M., Gaida, G. M., Lipman, J., Mishra, R., Goldman, H., Jessup, J. M., and Kolodner, R. Methylation of the hMLH1 promoter correlates with lack of expression of hMLH1 in sporadic colon tumors and mismatch repair-defective human tumor cell lines. Cancer Res., 57: 808-811, 1997.
7. Herman, J. G., Umar, A., Polyak, K., Graff, J. R., Ahuja, N., Issa, J. P., Markowitz, S., Willson, J. K., Hamilton, S. R., Kinzler, K. W., Kane, M. F., Kolodner, R. D., Vogelstein, B., Kunkel, T. A., and Baylin, S. B. Incidence and functional consequences of hMLH1 promoter hypermethylation in colorectal carcinoma. Proc. Natl. Acad. Sci. USA, 95:6870-6875, 1998.
8. Ahuja, N., Mohan, A. L., Li, Q., Stolker, J. M., Herman, J. G., Hamilton, S. R., Baylin, S. B., and Issa, J. P. Association between CpG island methylation and microsatellite instability in colorectal cancer. Cancer Res., 57: 3370-3374, 1997.
9. Shannon, B. A., and lacopetta, B. J. Methylation of the hMLH1, p16, and MDR1 genes in colorectal carcinoma: associations with clinicopathological features. Cancer Lett., 167: 91-97, 2001.
10. Wiencke, J. K., Zheng, S., Lafuente, A., Lafuente, M. J., Grudzen, C., Wrensch, M. R., Miike, R., Ballesta, A., and Trias, M. Aberrant methylation of p16 INK4a in anatomic and gender-specific subtypes of sporadic colorectal cancer. Cancer Epidemiol. Biomark. Prev., 8: 501-506, 1999.
11. Jass, J. R. Serrated route to colorectal cancer: back street or super highway? J. Pathol., 193: 283-285, 2001. 12. Hawkins, N., and Ward, R. Sporadic colorectal cancers with mic-rosatellite instability and their possible origin in hyperplastic polyps and serrated adenomas. J. Natl. Cancer Inst. (Bethesda), 93: 1307-1313,2001.
13. Elsaleh, H., Joseph, D., Grieu, F., Zeps, N., Spry, N., and lacopetta, B. Association of tumour site and sex with survival benefit from adjuvant chemotherapy in colorectal cancer. Lancet, 355: 1745-1750, 2000.
14. Elsaleh, H., Powell, B., McCaul, K., Grieu, F., Grant, R., Joseph, D., and lacopetta, B. P53 alteration and microsatellite instability have predictive value for survival benefit from chemotherapy in stage III colorectal carcinoma. Clin. Cancer Res., 7: 1343-1349, 2001.
15. Liang, J. T., Huang, K. C., Lai, H. S., Lee, P. H., Cheng, Y. M., Hsu, H. C., Cheng, A. L., Hsu, C. H., Yeh, K. H., Wang, S. M., Tang, C., and Chang, K. J. High-frequency microsatellite instability predicts better chemosensitivity to high-dose 5-fluorouracil plus leucovorin chemotherapy for stage IV sporadic colorectal cancer after palliative bowel resection. Int. J. Cancer, 101: 519-525,2002.
16. Taal, B. G., Van Tinteren, H., and Zoetmulder, F. A. Adjuvant 5FU plus levamisole in colonic or rectal cancer: improved survival in stage II and III. Br. J. Cancer, 85: 1437-1443, 2001.
17. Herman, J. G., Graff, J. R., Myohanen, S., Nelkin, B. D., and Baylin, S. B. Methylation-specific PCR: a novel PCR assay for methylation status of CpG islands. Proc. Natl. Acad. Sci. USA, 93: 9821-9826, 1996.
18. Iacopetta, B., and Grieu, F. Routine detection of the replication error phenotype in clinical tumor specimens using fluorescence-SSCP. Biotechniques, 28: 566-570, 2000.
19. Iacopetta, B., Elsaleh, H., Grieu, F., Joseph, D., Sterrett, G., and Robbins, P. Routine analysis of p53 mutation in clinical breast tumor specimens using fluorescence-based polymerase chain reaction and single strand conformation polymorphism. Diagn. Mol. Pathol., 9: 20-25, 2000.
20. Lin, D. Y., Psaty, B. M., and Kronmal, R. A. Assessing the sensitivity of regression results to unmeasured confounders in observational studies. Biometrics, 54: 948-963, 1998.
21. Moertel, C. G., Fleming, T. R., Macdonald, J. S., Haller, D. G., Laurie, J. A., Tangen, C. M., Ungerleider, J. S., Emerson, W. A., Tormey, D. C., Glick, J. H., et al. Fluorouracil plus levamisole as effective adjuvant therapy after resection of stage III colon carcinoma: a final report. Ann. Intern. Med., 122: 321-326, 1995.
22. Liang, J. T., Chang, K. J., Chen, J. C., Lee, C. C., Cheng, Y. M., Hsu, H. C., Wu, M. S., Wang, S. M., Lin, J. T., and Cheng, A. L. Hypermethylation of the p16 gene in sporadic T3 NO MO stage colorectal cancers: association with DNA replication error and shorter survival. Oncology (Basel), 57: 149-156, 1999.
23. Esteller, M., Tortola, S., Toyota, M., Capella, G., Peinado, M. A., Baylin, S. B., and Herman, J. G. Hypermethylation-associated inactivation of p14 ARF is independent of p16 INK4a methylation and p53 mutational status. Cancer Res., 60: 129-133, 2000.
24. Ahnen, D. J., Feigl, P., Quan, G., Fenoglio-Preiser, C., Lovato, L. C., Bunn, P. A., Jr., Stemmerman, G., Wells, J. D., Macdonald, J. S., and Meyskens, F. L., Jr. Ki-ras mutation and p53 overexpression predict the clinical behavior of colorectal cancer: a Southwest Oncology Group study. Cancer Res., 58: 1149-1158, 1998.
25. Sundararajan, V., Mitra, N., Jacobson, J. S., Grann, V. R., Heitjan, D. F., and Neugut, A. I. Survival associated with 5-fluorouracil-based adjuvant chemotherapy among elderly patients with node-positive colon cancer. Ann. Intern. Med., 136: 349-357, 2002.
26. Lenz, H. J., Hayashi, K., Salonga, D., Danenberg, K. D., Danenberg, P. V., Metzger, R., Banerjee, D., Bertino, J. R., Groshen, S., Leichman, L. P., and Leichman, C. G. p53 point mutations and thymidylate synthase messenger RNA levels in disseminated colorectal caner: an analysis of response and survival. Clin. Cancer Res., 4: 1243-1250, 1998.
27. Leichman, C. G., Lenz, H. J., Leichman, L., Danenberg, K., Baranda, J., Groshen, S., Boswell, W., Metzger, R., Tan, M., and Danenberg, P. V. Quantitation of intratumoral thymidylate synthase expression predicts for disseminated colorectal cancer response and resistance to protracted-infusion fluorouracil and weekly leucovorin. J. Clin. Oncol., 15:3223-3229, 1997.
28. Salonga, D., Danenberg, K. D., Johnson, M., Metzger, R., Groshen, S., Tsao-Wei, D. D., Lenz, H. J., Leichman, C. G., Leichman, L., Diasio, R. B., and Danenberg, P. V. Colorectal tumors responding to 5-fluorouracil have low gene expression levels of dihydropyrimidine dehydrogenase, thymidylate synthase, and thymidine phosphorylase. Clin. Cancer Res., 6: 1322-1327, 2000.
29. Choi, S. W., and Mason, J. B. Folate and carcinogenesis: an integrated scheme. J. Nutr., 130: 129-132, 2000.
30. Herman J G, Graff J R, Myohanen S, Nelkin B D, Baylin S B. Methylation-specific PCR: a novel PCR assay for methylation status of CpG islands. Proc Natl Acad Sci USA 1996; 93(18):9821-9826.
31. Eads C A, Danenberg K D, Kawakami K, Saltz L B, Blake C, Shibata D et al. MethyLight: a high-throughput assay to measure DNA methylation. Nucleic Acids Res 2000; 28(8):E32.
32. Holland et al; Detection of specific polymerase chain reaction product by utilising the 5′-3′ exonuclease activity of Thermus aquaticus DNA polymerase; Proc. Natl. Acad. Sci. USA 88, 7276-7280 (1991).
33. Gelmini et al. Quantitative polymerase chain reaction-based homogeneous assay with flurogenic probes to measure C-Erbb-2 oncogene amplification. Clin. Chem. 43, 752-758 (1997).
34. Livak et al. Towards fully automated genome wide polymorphism screening. Nat. Genet. 9, 341-342 (1995).
35. Tyagi & Kramer. Molecular beacons—probes that fluoresce upon hybridization. Nat. Biotechnol. 14, 303-308 (1996).
36. Tyagi et al. Multicolor molecular beacons for allele discrimination. Nat. Biotechnol. 16, 49-53 (1998).
37. David Whitcombe, Jane Theaker, Simon P. Guy, Tom Brown, Steve Little. Detection of PCR products using self-probing amplicons and fluorescence; Nature Biotechnology 17, 804-807 (1 Aug. 1999) 11.
38. Suzuki et al. Nature Genetics (2002) 31:141-149.
39. Smith and Gallagher, Eur J. Cancer (2003) 39:1377-1383
40. Droz et al., Ann. Oncol. (2003) 14:1291-1298
41. Cocconi et al. Ann. Oncol. (2003) 14:1258-1263
42. F. G., J. Obset. Gyneaecol. (2003) 23:422-425
43. Focan et al., Pathol Biol (Paris) (2003) 51:204-205
44. Lee et al., Acta Oncol. (2003) 42:207-217.
45. Compton. Nucleic acid sequence-based amplification. Nature 7; 350(6313): p91-92 (March 1991)
46. Fahy et al. Self-sustained sequence replication (3SR): an isothermal transcription-based amplification system alternative to PCR. PCR Methods Appl. 1(1): p25-33 (August) 1991

1. A method for predicting the likelihood of successful treatment of cancer with an antimetabolic compound comprising measuring the CIMP status of a sample obtained from a subject, whereby if the CIMP status is positive the likelihood of successful treatment is higher than if the CIMP status is negative.

2. A method of selecting a suitable treatment regimen for cancer comprising determining the CIMP status of a sample obtained from a subject, whereby if the CIMP status is positive treatment using antimetabolic compounds may be administered.

3. The method according to claim 1 or 2 wherein the cancer is selected from colorectal cancer (CRC), pancreatic cancers, breast cancers, prostate cancers, gastric cancers, Cervix cancers, lung cancers, esophageal cancers, Renal cancers, head and neck cancers.

4. The method according to claim 3 wherein the cancer is colorectal cancer (CRC).

5. The method according to any one of claims 1 to 4 wherein the antimetabolic compound is a inhibitor of cancer cell metabolism, nucleotide and DNA metabolism, or methylation, purine, methyl group metabolism, folate metabolism or folate in nucleic acid metabolism.

6. The method according to any one of claims 1 to 5 wherein the antimetabolic compound is a inhibitor of Thymidilate synthase, dihydrofolate reductase, AICAR transformylase, GAR transformylase, several methyl transferases, methylenetetrahydrofolate reductase, DNA polymerase adenosine deaminase, methionine synthase, and/or cystathionine-beta-synthase.

7. The method according to claim 6 wherein the antimetabolic compound is a Thymidylate synthase (TS) inhibitor.

8. The method according to claim 7 wherein the Thymidylate synthase (TS) inhibitor comprises a folate analogue or a nucleotide analogue selected from:
5-FU, fluorodeoxyuridine, ftorfur, 5′-deoxyfluoruridine, raltitrexed, UFT, S-1, 5-ethynyluracil, Capecitabine, pemetrexed, nolatrexed, ZD9331, trimetrexate, LU231514, edatrexate, GW1843, GW1843, OSI-7904L, Leucovorin, Levimosole, Methotrexaate, GS7904L, PDX, 10-EdAM, ICI-198,583 and DDATHF; CB300638, 4-S-CAP and N-ac-4-S-CAP.

9. The method according to claim 5 wherein the antimetabolic compound used is an inhibitor of folate metabolic pathways selected from: Cytarabine (Ara-C) and Gemcitabine, 6-MP and 6-TG (thiopurines), Fluarabine, Cladribine and Pentostatin.

10. The method according to any one of claims 1 to 9 wherein the CIMP status is measured by determining the methylation status of a panel of genes.

11. The method according to claim 10 wherein the panel of genes comprises at least two genes from: THBS1, IGF-2, HIC-1 and hMLH1, p16, MINT-2, MDR1, p15, E-cadherin, VHL, TGFβ1, TGFβ2, P130, BRAC2, NF1, NF2, TSG101, MDGI, GSTPI, Calcitonin, HIC-1, Endothelin B receptor, TIMP-2, MGMT, MLH1, MLH2 and GFAP; MGMT, DAP kinase, RASSF1A, H-cadherin, retinoic acid receptor beta, and fragile histidine triade; TSLC1; SOCS-1 SOCS-2, CIS-2; APC, DAPK, PAX5 alpha, PAX5 beta, Gata-4, Gata-5, Dab-2, inhibin α, Tiff2, and Tiff3, AP-2 α, P73, BRAC-1, RASSF-1, P14, E-cadherin, RARbeta2, TIMP3, CDH1, BRAC-1, and Tromb.

12. The method according to claim 10 or 11 wherein the panel of genes comprises the following genes: p16, MINT-2 and MDR1.

13. The method according to any one of claims 10 to 12 wherein the CIMP status will be considered positive if all, or at least 2, or at least 3, or at least 4, or at least 5, or at least 6 of the promoters of the panel of genes are methylated.

14. The method according to any one of claims 10 to 13 wherein the methylation status of the genes is measured using methylation specific PCR (MSP) or quantitative methylation specific PCR (QMSP).

15. The method according to any one of claims 10 to 14 wherein the methylation status of the panel of genes is determined in a single experiment.

16. The method according to any one of claims 10 to 14 wherein the methylation status of each of the panel of genes is determined in a separate experiment.

17. The method according to any one of claims 1 to 16 wherein the CIMP status is measured or additionally measured by determining the expression of a panel of genes at either the RNA or protein level.

18. The method according to claim 17 wherein the panel of genes comprises at least two genes from: THBS1, IGF-2, HIC-1 and hMLH1, p16, MINT-2, MDR1, p15, E-cadherin, VHL, TGFβ1, TGFβ2, P130, BRAC2, NF1, NF2, TSG101, MDGI, GSTPI, Calcitonin, HIC-1, Endothelin B receptor, TIMP-2, MGMT, MLH1, MLH2 and GFAP; MGMT, DAP kinase, RASSF1A, H-cadherin, retinoic acid receptor beta, and fragile histidine triade; TSLC1; SOCS-1 SOCS-2, CIS-2; APC, DAPK, PAX5 alpha, PAX5 beta, Gata-4, Gata-5, Dab-2, inhibin a, Tiff2, and Tiff3, AP-2 α, P73, BRAC-1, RASSF-1, P14, E-cadherin, RARbeta2, TIMP3, CDH1, BRAC-1, and Tromb.

19. The method according to claim 17 or 18 wherein the panel of genes comprises the following genes: p16 and hMLH1.

20. The method according to any one of claims 1 to 19 additionally comprising measuring expression levels of genes involved in folate metabolism.

21. The method according to claim 20 wherein the genes involved in folate metabolism comprise any of the genes encoding thymidylate synthetase, dihydropyrimidine dehydrogenase and thymidine phosphorylase.

22. The method according to any one of claims 1 to 21 additionally comprising measuring the levels of genomic hypomethylation.

23. The method according to claim 22 wherein the genes comprise any of: claudin4, lipocalin2, 14-3-3sigma, trefoil factor2, S100A4, mesothelin, prostate stem cell antigen, CAGE, methyltransferases (DNMT1, 3A and 3B), MYOD1, Synuclein Gamma (SNCG, BCSG1), MUC2, H19, IGF2, CDH13,

24. The method according to claim 22 or 23 wherein levels of genomic hypomethylation are measured using MSP or QMSP.

25. The method according to any one of claims 1 to 24 additionally comprising measuring the levels of intra-tumoral folate intermediates.

26. The method according to claim 2 wherein if the CIMP status is positive treatment using antimetabolic compounds will be used in conjunction with surgical techniques.

27. The method according to claim 2 wherein if the CIMP status is negative surgical techniques will be utilised possibly in conjunction with other chemotherapies, other than treatment using antimetabolic compounds.

28. A kit for predicting the likelihood of successful treatment of cancer with antimetabolic compounds comprising
means for measuring the CIMP status of a sample obtained from a subject; and
means for contacting the sample with the means for measuring CIMP status.

29. The kit according to claim 28 wherein the means for measuring the CIMP status of a sample includes means for determining the methylation status of the promoters of a panel of genes.

30. The kit according to claim 29 wherein the panel of genes comprises any number of the following genes: THBS1, IGF-2, HIC-1 and hMLH1, p16, MINT-2, MDR1, p15, E-cadherin, VHL, TGFβ1, TGFβ2, P130, BRAC2, NF1, NF2, TSG101, MDGI, GSTPI, Calcitonin, HIC-1, Endothelin B receptor, TIMP-2, MGMT, MLH1, MLH2 and GFAP; MGMT, DAP kinase, RASSF1A, H-cadherin, retinoic acid receptor beta, and fragile histidine triade; TSLC1; SOCS-1 SOCS-2, CIS-2; APC, DAPK, PAX5 alpha, PAX5 beta, Gata-4, Gata-5, Dab-2, inhibin α, Tiff2, and Tiff3, AP-2 α, P73, BRAC-1, RASSF-1, P14, E-cadherin, RARbeta2, TIMP3, CDH1, BRAC-1, and Tromb.

31. The kit according to claim 30 wherein the panel of genes comprises the following genes: p16, MINT-2 and MDR1.

32. The kit according to any of claims 28 to 31 including MSP and/or QMSP reagents to measure the CIMP status of the sample.

33. The kit according to claim 32 including gene specific primers for the genes whose methylation status is measured in order to determine CIMP status.

34. The kit according to any one of claims 28 to 33 further containing gene specific probes and reagents to allow real-time detection of QMSP reaction products.

35. The kit according to claim 34 wherein the real-time detection method is selected from Taqman system, Molecular beacons system and Scorpion probe system.

36. The kit according to any one of claims 28 to 35 comprising, or additionally comprising, means for determining the expression of a panel of genes at either the RNA or protein level in order to measure CIMP status.

37. The kit according to claim 36 wherein the panel of genes comprises any number of the following genes: THBS1, IGF-2, HIC-1 and hMLH1, p16, MINT-2, MDR1 p15, E-cadherin, VHL, TGFβ1, TGFβ2, P130, BRAC2, NF1, NF2, TSG101, MDGI, GSTPI, Calcitonin, HIC-1, Endothelin B receptor, TIMP-2, MGMT, MLH1, MLH2 and GFAP; MGMT, DAP kinase, RASSF1A, H-cadherin, retinoic acid receptor beta, and fragile histidine triade; TSLC1; SOCS-1 SOCS-2, CIS-2; APC, DAPK, PAX5 alpha, PAX5 beta, Gata-4, Gata-5, Dab-2, inhibin α, Tiff2, and Tiff3, AP-2 α, P73, BRAC-1, RASSF-1, P14, E-cadherin, RARbeta2, TIMP3, CDH1, BRAC-1, and Tromb.

38. The kit according to claim 35 or 36 wherein the panel of genes comprises the following genes: p16 and hMLH1.

39. The use of antimetabolic compounds in the treatment of a subject suffering from cancer, wherein said subject has a positive CIMP status.

40. Antimetabolic compounds for use in the manufacture of a medicament for the treatment of a subject suffering from cancer, wherein said subject has a positive CIMP status.


SEQUENCE LISTING (2 sequences)

SEQ ID NO: 1 - DNA, 21 bp, Artificial sequence
  Primer
ttaataggaagagcggatagc

SEQ ID NO: 2 - DNA, 23 bp, Artificial sequence
  Primer
ctataaattactaaatctcttcg